(19) United States
(12) Patent Application Publication
(10) Pub. No.: US 2017/0245729 A1
(43) Pub. Date: Aug. 31, 2017

(54) DISHWASHER CONTAINER FOR BREAST PUMP PARTS

(71) Applicant: Pumpie LLC, St. Louis, MO (US)

(72) Inventors: Helen M. Ciesielski, St. Louis, MO (US); Melvin J. Leedle, Bridgeton, MO (US)

(73) Assignee: Pumpie LLC, St. Louis, MO (US)

(21) Appl. No.: 15/442,912

(22) Filed: Feb. 27, 2017

Related U.S. Application Data
(60) Provisional application No. 62/300,950, filed on Feb. 29, 2016.

(51) Int. Cl. A47L 15/50; B65D 33/25; B65D 33/28; A61M 1/06; B65D 6/16; B65D 6/02; B65D 8/00; B65D 30/22; B65D 25/06

(52) U.S. Cl. CPC A47L 15/505; B65D 31/12; B65D 33/25; B65D 33/28; B65D 25/06; B65D 11/18; B65D 7/065; B65D 15/22; A61M 1/062; A61M 2209/10

(57) ABSTRACT

A dishwasher container that can hold breast pump parts. The container can include a bottom fabric panel, a pair of side fabric panels, a divider panel, and a closure. The panels can include flexible fabric strands and spaces between the flexible fabric strands. The pair of side fabric panels can be connected to the bottom fabric panel at a bottom seam around a circumference of the bottom fabric panel and extending away from the bottom fabric panel to an opening at a top end. The divider fabric panel can be within an interior space formed by the bottom and side panels. The closure can enclose the interior space when fastened.

### CROSS REFERENCE TO RELATED APPLICATIONS

This patent application claims the benefit of U.S. Provisional Patent Application No. 62/300,950, filed Feb. 29, 2016, entitled “DISHWASHER CONTAINER FOR BREAST PUMP PARTS,” which is incorporated herein by reference in its entirety.

### BACKGROUND

Field

Embodiments described herein generally relate to a container for holding components, including holding components in a dishwasher during cleaning, and more particularly to an improved container for holding one or more components of a breast pump assembly in a dishwasher during cleaning.

Related Art

Breast pump and nursing bottle assemblies are commonly used for extracting milk from a mother's breasts and feeding infants. The breast pump typically includes parts that can be removed from the pump and washed separately, such as the breast shield, connectors, valves, membranes, and caps. Feeding bottle assemblies typically include the bottle and other components such as a nipple, collar, and a cap. To clean the assemblies, the various parts and components of the assemblies are disassembled and separately loaded into a standard dishwasher.

Usually, the breast pump parts and bottle components other than the bottle are placed in a dishwasher, such as a utensil basket, where the parts and components are free to rotate and move relative to each other, and the bottles to be placed on the upper rack of the dishwasher along with glasses and cups. Loading the pump parts and bottle components into a utensil basket in this manner can result in a less than desired cleaning of the parts and components, which can lead to the spread of disease and potentially harm the infant and/or mother. For example, some parts and components, upon loading or upon movement in the basket during cleaning, end up being surrounded or otherwise blocked by the other parts and components in the basket to the point that the blocked pieces are not cleaned well. It is also possible that some of pieces may become nested within other pieces in the basket, blocking the internal sides of the nested pieces from being cleaned. Additionally, it is possible for the pieces to be oriented such that they collect water and the sediment that is contained within the water.

Previous dishwasher safe containers have been extremely complex and include complex parts or are form-fitting to proprietary part designs which result in costly containers with limited functionality. Further, many of these containers are bulky so they are not useful for carrying the parts even when they have form-fitting designs. Some previous containers have thick walls that disadvantageously block the water from cleaning the parts, and/or have large opening that allow the smallest parts of the breast pump (e.g., the membranes) to exit the container during the dishwasher cycle. Therefore, there remains a need for a container that holds breast pump and bottle parts separate from each other during cleaning so that the contained parts are sufficiently cleaned, while reducing the complexity and expense to manufacture and are easy to use. Examples of known devices are described in the references listed below, which are hereby incorporated by reference.

| | | | |
|---|---|---|---|
| U.S. Pat. No. 2,602,482 | U.S. Pat. No. 5,092,682 | U.S. Pat. No. 5,102,225 | U.S. Pat. No. 5,211,191 |
| U.S. Pat. No. 6,371,642 | U.S. Pat. No. 6,814,091 | U.S. Pat. No. 6,910,488 | U.S. Pat. No. 6,915,808 |
| U.S. Pat. No. 7,628,163 | U.S. Pat. No. 7,650,896 | US2007/0014493 | |

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1A illustrates a prospective view of a mesh bag container in an open position taken generally from the top and side thereof according to an exemplary embodiment of the present disclosure.

FIG. 1B illustrates a prospective view of the mesh bag container of FIG. 1A taken generally from the side and bottom thereof.

FIG. 1C illustrates a bottom elevation view of the mesh bag container of FIG. 1A.

FIG. 1D illustrates a prospective view of the mesh bag container of FIG. 1A in a closed position taken generally from the side and top thereof.

FIG. 1E illustrates a top view of the mesh bag container of FIG. 1A in the open position.

FIG. 1F illustrates the mesh bag container of FIG. 1D having example articles contained therein taken generally from the side and top thereof.

FIG. 1G illustrates a side end view of the mesh bag container of FIG. 1F.

FIG. 1H illustrates example breast pump parts that can be contained with the containers of one or more exemplary embodiments of the present disclosure.

FIG. 2A illustrates a prospective view of a mesh bag container in a closed position taken generally from the top and side thereof according to another exemplary embodiment of the present disclosure.

FIG. 2B illustrates a top view of the mesh bag container of FIG. 2A in an open position.

FIG. 2C illustrates a prospective view of the mesh bag container of FIG. 2A in a closed position taken generally from the side and top thereof.

FIG. 2D illustrates a side elevational view of the mesh bag container of FIG. 2A.

FIG. 2E illustrates a top view of the mesh bag container of FIG. 2A in an opened position.

FIG. 2F illustrates a top view of the mesh bag container of FIG. 2A in a closed position.

FIG. 2G illustrates a side end elevational view of the mesh bag container of FIG. 2A in a closed position.

FIG. 2H illustrates a bottom view of the mesh bag container of FIG. 2A.

FIG. 3A illustrates a prospective view of a container in a closed position taken generally from the top, front, right-side thereof according to another exemplary embodiment of the present disclosure.

FIG. 3B illustrates a prospective view of the container of FIG. 3A in an opened position taken generally from the top, front, right-side thereof.

FIG. 3C illustrates a front elevational view of the container of FIG. 3A.

FIG. 3D illustrates a right side elevational view of the container of FIG. 3A.

FIGS. 4A-4B illustrate prospective views of a container in a closed position taken generally from the top, front, left-side thereof according to another exemplary embodiment of the present disclosure.

The exemplary embodiments of the present disclosure will be described with reference to the accompanying drawings.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure.

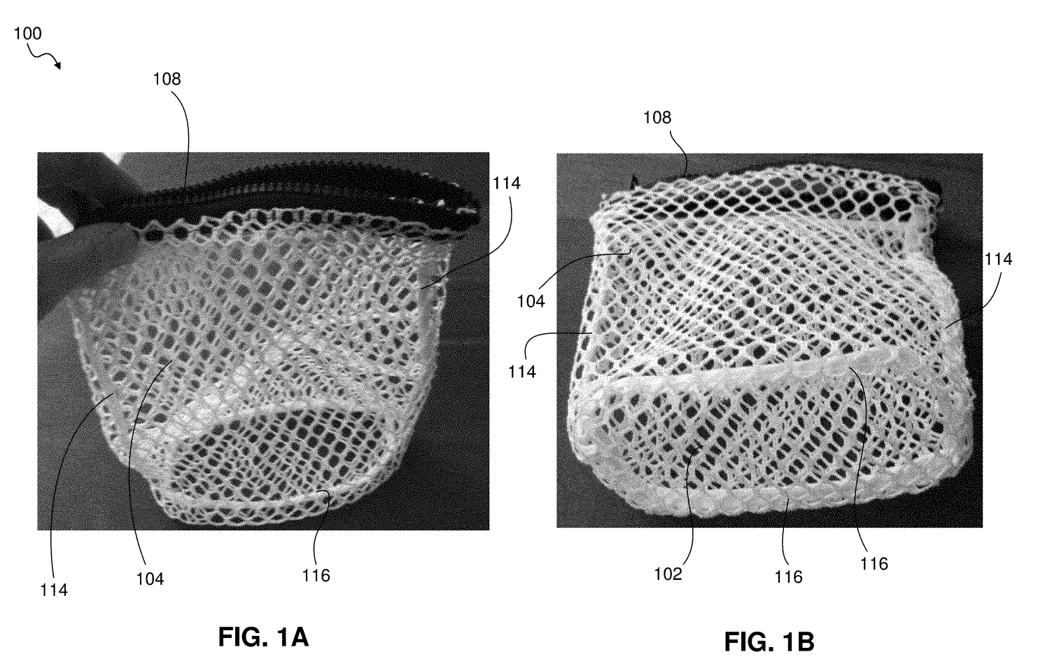

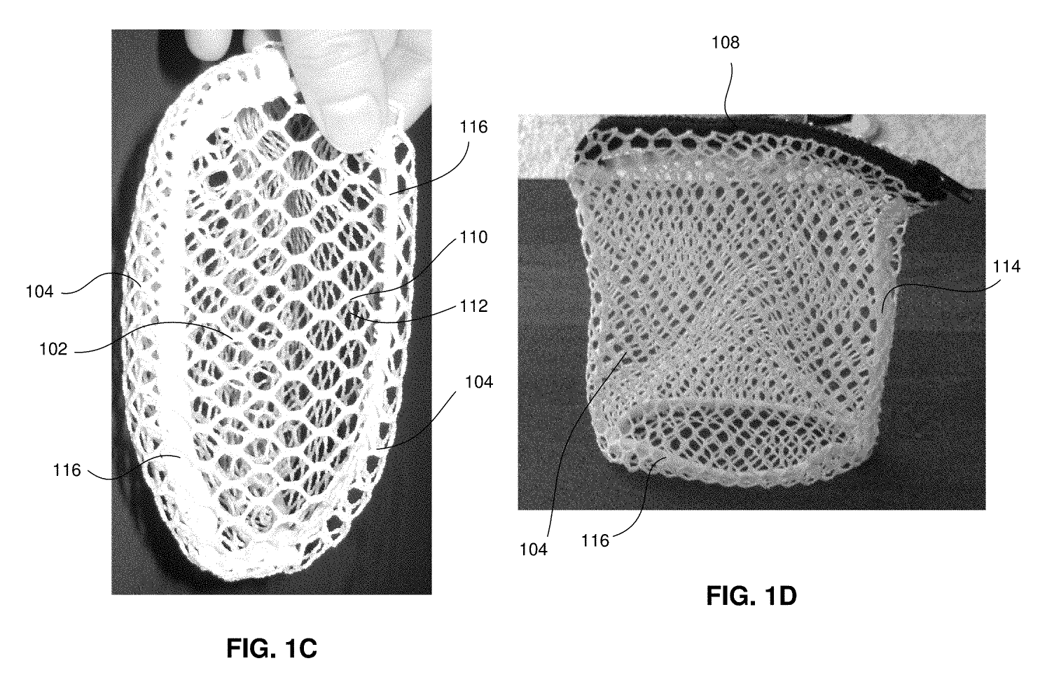

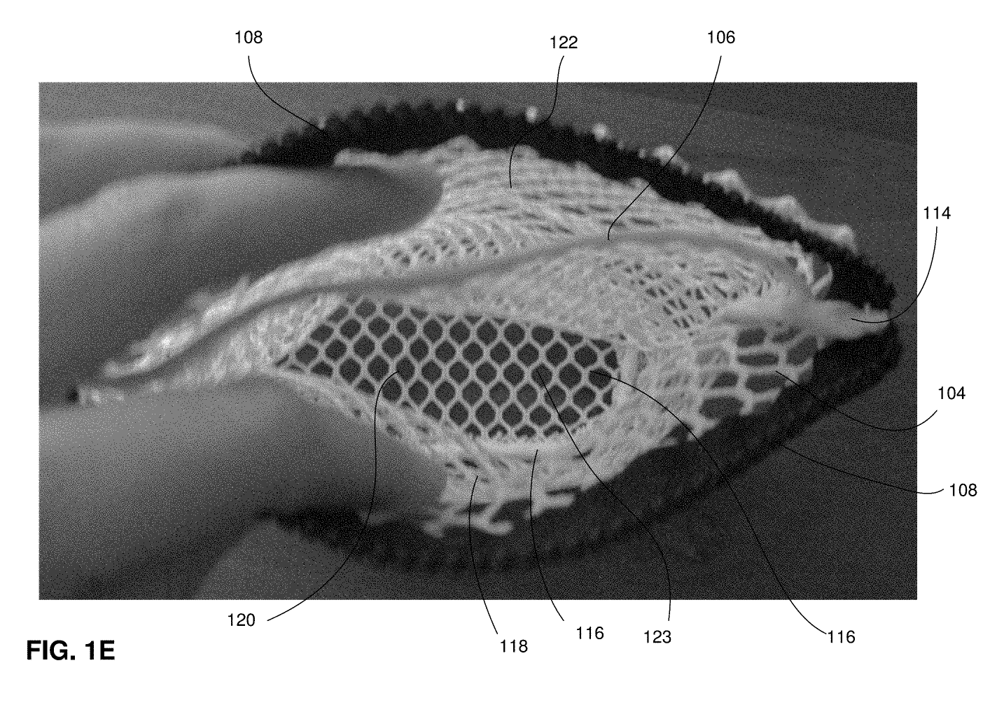

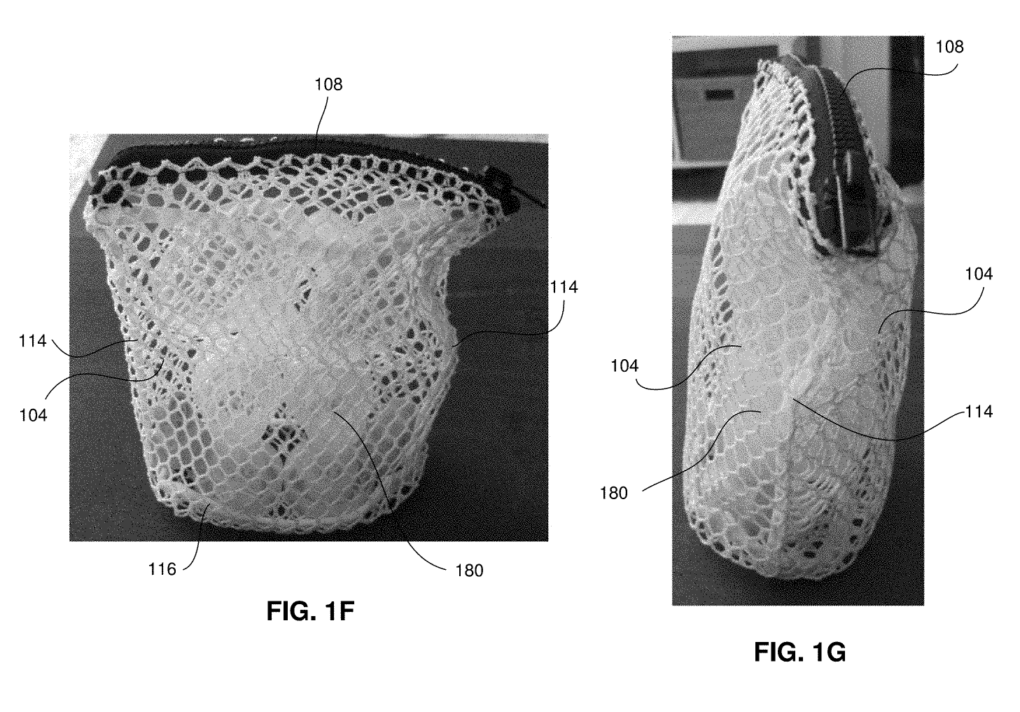

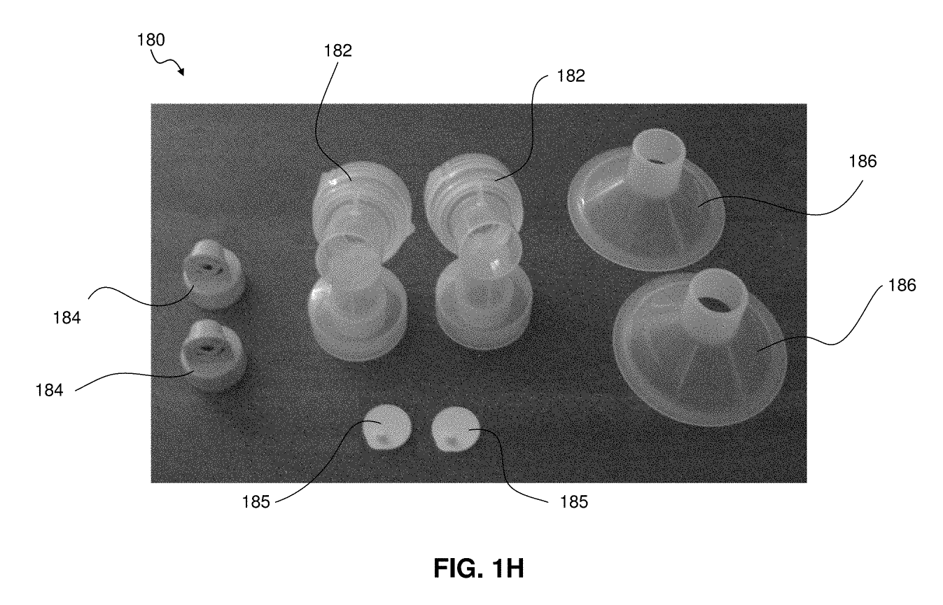

FIGS. 1A-1G illustrate a mesh bag container 100 according to an exemplary embodiment of the present disclosure. The mesh bag container 100 is configured to hold one or more articles, such as one or more breast pump parts 180 (see FIG. 1H). For example, the mesh bag container 100 can be configured to hold one or more breast pump parts 180 for a washing in a dishwashing device, such as a dishwasher.

In an exemplary embodiment, the mesh bag container 100 has a bottom fabric panel 102, a pair of side fabric panels 104, a divider fabric panel 106, and a closure mechanism 108. In an exemplary embodiment, the closure mechanism 108 is a zipper, but is not limited thereto. The closure mechanism 108 can be, for example, a snap clasp, a draw string, a hook-and-loop fastener, a magnetic clasp, a button and hole fastening arrangement, or another fastener as would be understood by one of ordinary skill in the art.

The fabric panels 102, 104 and/or the divider fabric panel 106 can be formed from pieces of mesh fabric that have flexible fabric strands 110 and a plurality of spaces (e.g., apertures) 112 between the flexible fabric strands 110. In an exemplary embodiment, the spaces 112 are slightly smaller than the diameter of the smallest breast pump part 180, the membranes. In an exemplary embodiment, the spaces 112 have an opening with the dimensions of, for example, between 0.2 inches and 0.45 inches, but is not limited thereto. The spaces 112 can have other dimensions as would understood by one of ordinary skill in the art such that the contained parts 180 are exposed to the washing fluid while securely containing the parts 180 within the container 100.

In an exemplary embodiment, the fabric panels 102, 104 and/or the divider fabric panel 106 are formed from a mildew-resistant, quick-drying textile, such as, for example, polyester, nylon or other synthetic fibers, but are not limited thereto. As would be understood by one of ordinary skill in the relevant arts, different types of knits and constructions can be used to form the fabric panels 102, 104 and/or the divider fabric panel 106, and the exemplary embodiment has hexagonal spaces 112 to maximize the flow-through area in the fabric.

In an exemplary embodiment, the container 100 can be configured as a rigid or semi-rigid structure similar to containers 300 and/or 400 described below. In this example, the container 100 can be made of, for example (but not limited to), plastic, metal, a combination of plastic and metal, and/or one or more other materials or composites as would be understood by one of ordinary skill in the art. For example, panels 102, 104, and/or 106 can be formed as a rigid or semi-rigid structure (e.g., a plastic panel). In an exemplary embodiment, the container 100 can be formed with a combination of rigid/semi-rigid panels and fabric panels. For example, panels 102 and 104 can be formed as rigid plastic panels and the divider panel 106 can be formed as a fabric panel.

In an exemplary embodiment, the bottom fabric panel 102 is approximately twice as long as it is wide. That is, the bottom fabric panel 102 has a longitudinal size greater than the lateral size, and side seams 114 are located at opposite longitudinal ends, on the long sides. The side fabric panels 104 are connected to the bottom fabric panel 102 at a bottom seam 116 around a circumference of the bottom fabric panel 102 and extend away from the bottom fabric panel 102 to an opening 118 at a top end. The opening 118 can be opened and closed using the closure mechanism 108.

The side fabric panels 104 are connected to each other through a pair of side seams 114 that extend from the bottom seam 116 to the top end between the pair of side fabric panels. The pair of side fabric panels 104 and the bottom fabric panel 102 define an interior space 120, and the divider fabric panel 106 is secured within the interior space 120 and is configured to divide the interior space 120 into two or more interior sections 122, 123 between the pair of side fabric panels 104. The divider fabric panel 106 extends from the bottom of the interior space 120 (i.e., proximate to the bottom fabric panel 102) to the top of the interior space 120 proximate to the opening 118 and the closure mechanism 108 at the top end of the container 100. The divider fabric panel 106 is connected to the pair of side fabric panels 104 at the pair of sides through the pair of side seams 114. The divider fabric panel 106 can divide the interior 120 of the container 100 in a longitudinal plane relative to the bottom panel 102. In an exemplary embodiment, the container 100 is configured such that the side seams 114 and/or the bottom seam 116 are formed on the exterior of the container 100. In another embodiment, the side seams 114 and/or the bottom seam 116 are formed in the interior of the container 100 (e.g., within the interior space 120.

When the parts 180 are placed in the container 100, its width may bulge out very slightly from the width of the bottom panel 102. In an exemplary embodiment, the length and width of the bottom panel is generally sized for a single set of breast pump parts 180 to be placed adjacent to each other, but can be sized for multiple sets of parts 180 in other embodiments. In an exemplary embodiment, two connectors 182 can be situated adjacent to each other at the bottom of the mesh bag 100 with the corresponding valve assemblies 184 and membranes 185, and the height of the side fabric panels 104 allows for two breast shields 186 to be placed adjacent to each other over the connectors 182. In an exemplary embodiment, the breast shields 186 can be placed on the opposite side of the divider panel 106 with respect to the connectors 182 and/or valve assemblies 184 to keep the parts from being displaced or knocked over during the dishwasher cycle.

In an exemplary embodiment, the closure mechanism 108 is secured to the opening 118 at the top end of the container 100. The closure mechanism 108 can have a fastened (i.e., closed) configuration enclosing the interior space 120 and have an unfastened (i.e., opened) configuration allowing access to the interior space 120 though the opening 118. The closure mechanism 108 can hold the parts 180 within the interior space 120 of the container 100 by pulling the top ends of the side fabric panels 104 together and toward the divider fabric panel 106 (e.g., at the center of the container 100) which secures the parts 180 from moving within the container 100 during the dishwasher cycles. In an exemplary embodiment, the closure mechanism 108 is a zipper as shown in FIGS. 1A-1G, but is not limited thereto. In an exemplary embodiment, the closure mechanism 108 is oriented lengthwise along the longitudinal axis of the bottom panel 102 and is aligned with the longitudinal plane of the divider panel 106.

In an exemplary embodiment, the mesh bag container 100 is configured to have structural support so that its flat bottom panel 102 can stand the container 100 upright on a dishwasher rack. The bottom seam 116, side seams 114, and/or the divider fabric panel 106 are configured to increase the structural support of the container 100 so that the flexible fabric forming the container 100 does not collapse on itself and block the water jets that clean the parts within the bag 100 during the dishwasher cycles. In an exemplary embodiment, the side seams 114 are formed with, for example, bias tape wrapped around the ends of the mesh fabric (of the panels 104, or panels 104 and 106) and sewn together which provides a greater rigidity than the side fabric panels 104 alone. Additionally, the divider fabric panel 106 provides additional rigidity and support between the side seams 114 and the side fabric panels 104. In an exemplary embodiment, it is also possible for the side seams 114 to include a bunching of mesh fabric to provide additional support. When the parts 180 held within the interior space 120 of the mesh bag container 100 and they are in the dishwasher cycle, the parts 180 provide substantial support to the mesh panels 104 and/or 106, and the seams 114 provide additional support to help prevent the panels 104 and/or 106 from being pushed into the spaces formed between the parts 180 and folding over on themselves, which could block water from reaching the parts 180 to be cleaned.

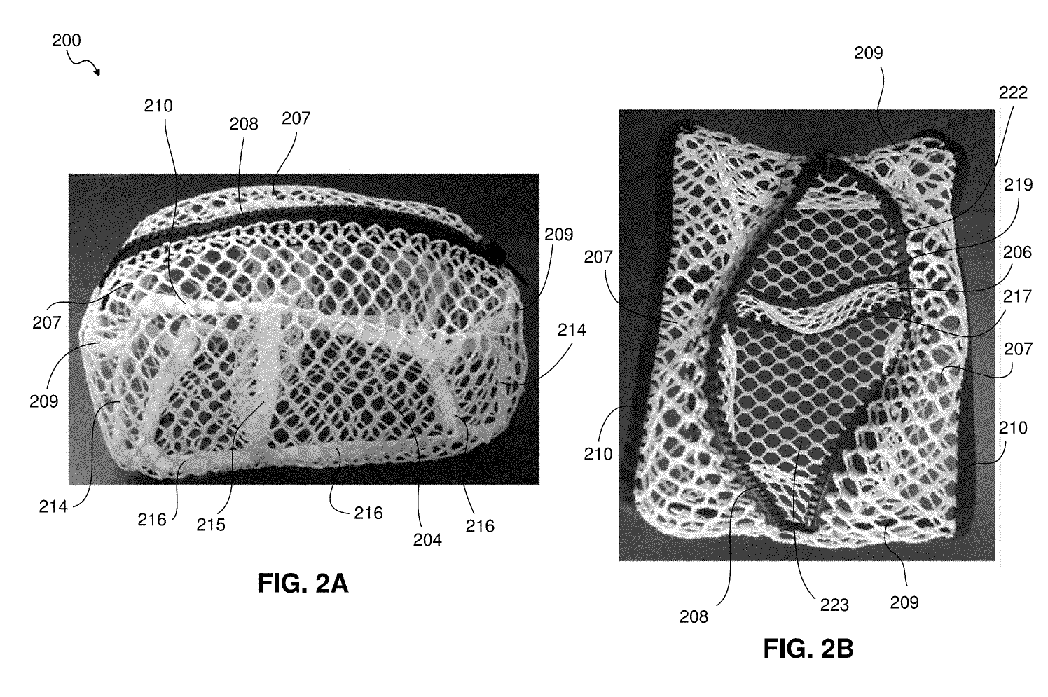

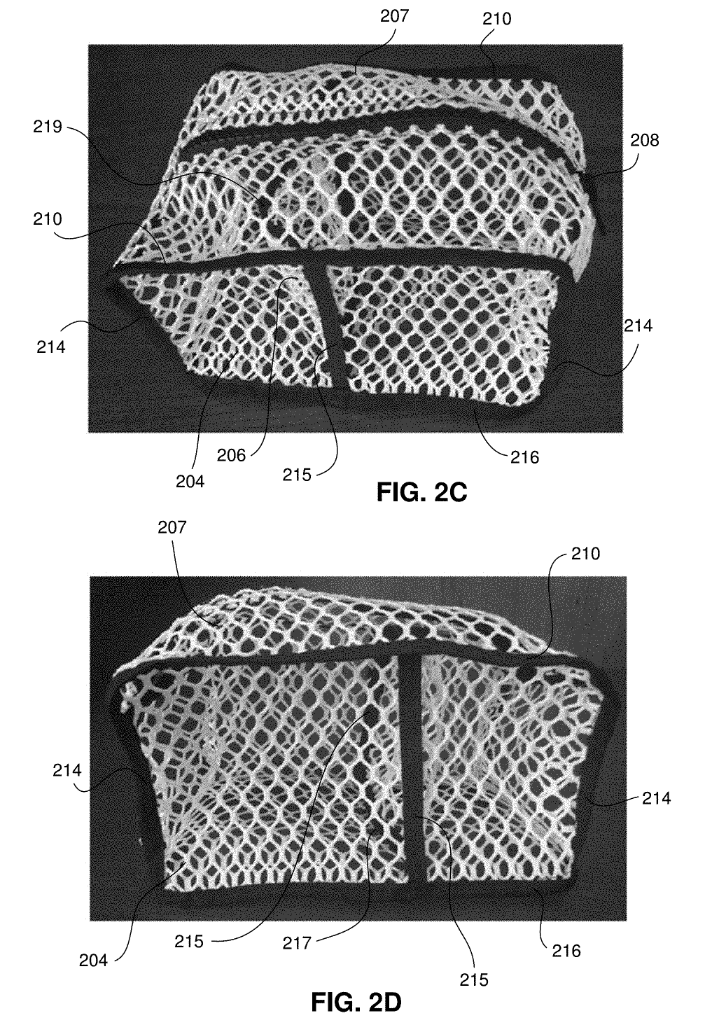

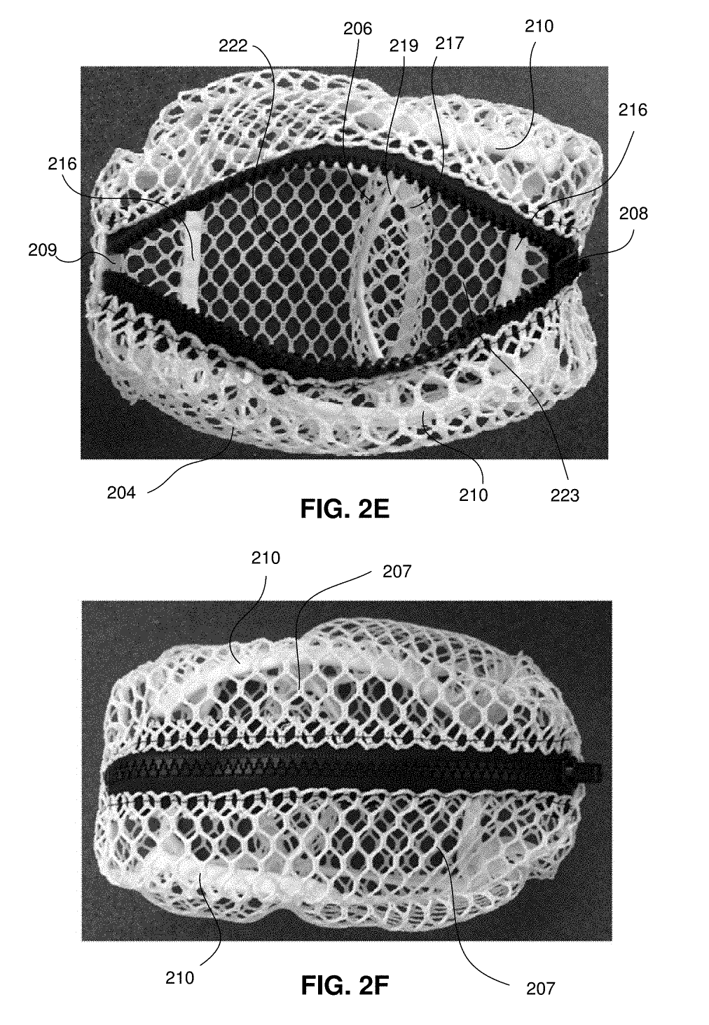

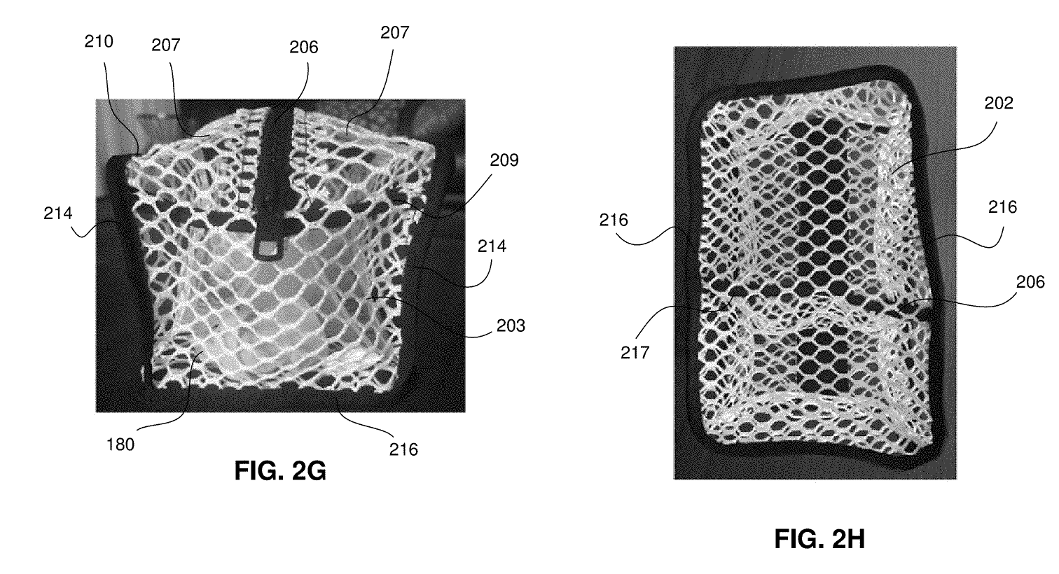

FIGS. 2A-2H illustrate a mesh bag container 200 according to another exemplary embodiment of the present disclosure. The container 200 is similar to the container 100 but has a shorter height and is wider than the container 100 of FIG. 1. The container 200 can have many of the same structural features as the container 100, including the mesh panel construction with side seams providing additional structural support. For example, the container 200 can include fabric panels formed from a mildew-resistant, quick-drying textile, such as, for example, polyester, nylon or other synthetic fibers, but are not limited thereto. Additionally, the container 200 can include a divider panel 206 which divides the interior 220 of the mesh bag 200 into two interior sections 222, 223 to help keep the parts 180 in their proper place and orientation.

In an exemplary embodiment, as with the container 100, the divider panel 206 provides additional support to the mesh bag container 200 as well as keeping the parts 180 from being displaced or knocked over.

In an exemplary embodiment, the container 200 includes an additional pair of side seams 215 that connect the divider panel 206 to opposing side panels 204. The divider panel 206 can be located between end panels 203. The divider panel 206 can be positioned such that the interior sections 222, 223 are of equal size or of different sizes. That is, one interior section 222 can be larger than another section 223 to more securely hold a single set of breast pump parts. For example, the connectors 182 can be held in a vertical orientation (e.g., as shown in FIG. 1F) in the smaller interior section 223. The larger interior section 222 can hold the breast shields 186, and the valves 184 and membranes 185 can fit in the remaining spaces between the connectors 182 and/or the breast shields 186. In this embodiment, the divider panel 206 extends across the bottom panel 202 in a lateral plane such that the longitudinal axis of the closure is generally perpendicular to the divider panel 206, and an optional intermediate bottom seam 217 may connect the bottom side of the divider panel 202 to the bottom panel. The divider panel 202 can also include an additional seam 219 on the top of the divider panel 202 to provide additional support.

In an exemplary embodiment, to give the container 200 additional support for its extra width, the container 200 can include two side seams 214 at each longitudinal end of the bottom panel 202 rather than a single side seam (e.g., 114 in container 100) at each longitudinal end as in the embodiments of container 100. The end panels 203 are connected to the side panels through the side seams 214 at each longitudinal end of the bottom panel 202, and a top lateral seam 209 connects the side seams 214 at the top of each longitudinal end. In an exemplary embodiment, the lateral seams 209 are formed in the interior portion of the mesh bag 200 as shown. In another embodiment, the lateral seams 209 are formed on the exterior of the mesh bag 200.

In an exemplary embodiment, the container 200 can include a pair of top longitudinal seams 210 that connects a top panel 207 to the side panel 204, and the closure mechanism 208 is secured to an opposite side of the top panel 207. In an exemplary embodiment, the side panels 204 can wrap around the ends of the bottom panel 202 and the top panel 207 could wrap around to the end panels 203. Accordingly, it is possible to produce the mesh bag 200 with two pieces of mesh fabric connected by the seams (e.g., 209, 210, 214, 216, 219) and the additional seams (e.g., 215, 217) can bunch the sections of the remaining panels to provide support to the mesh bag container 200.

In an exemplary embodiment, the container 200 can be configured as a rigid or semi-rigid structure similar to containers 300 and/or 400 described below. In this example, the container 200 can be made of, for example (but not limited to), plastic, metal, a combination of plastic and metal, and/or one or more other materials or composites as would be understood by one of ordinary skill in the art. For example, panels 202, 203, 204, 206, and/or 207 can be formed as a rigid or semi-rigid structure (e.g., a plastic panel). In an exemplary embodiment, the container 200 can be formed with a combination of rigid/semi-rigid panels and fabric panels. For example, panels 202, 203, 204, and/or 207 can be formed as rigid plastic panels and the divider panel 206 can be formed as a fabric panel.

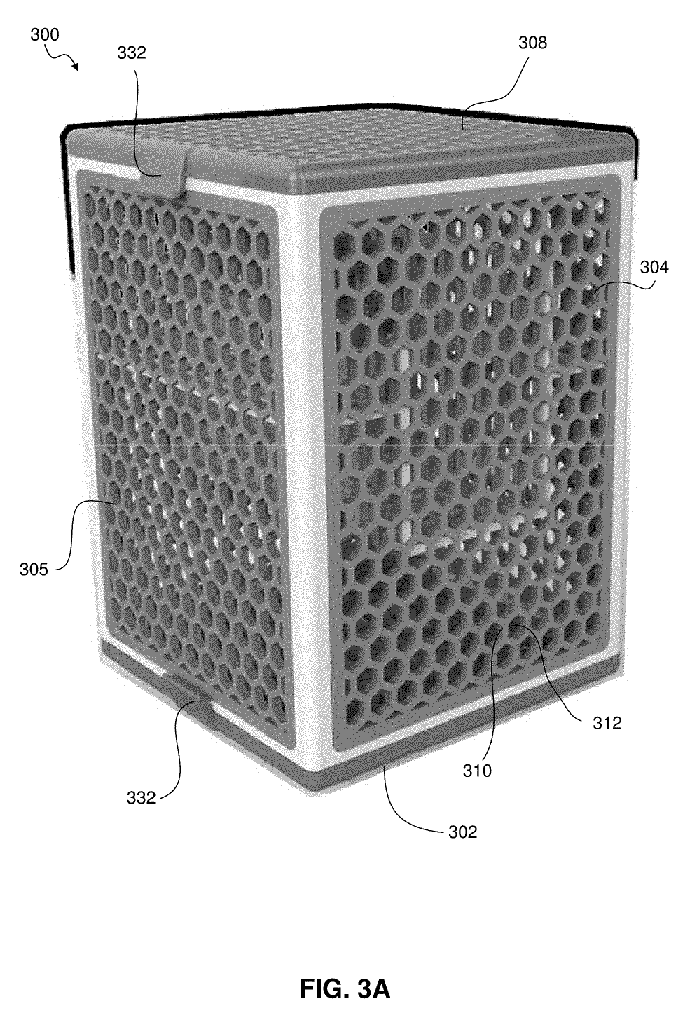

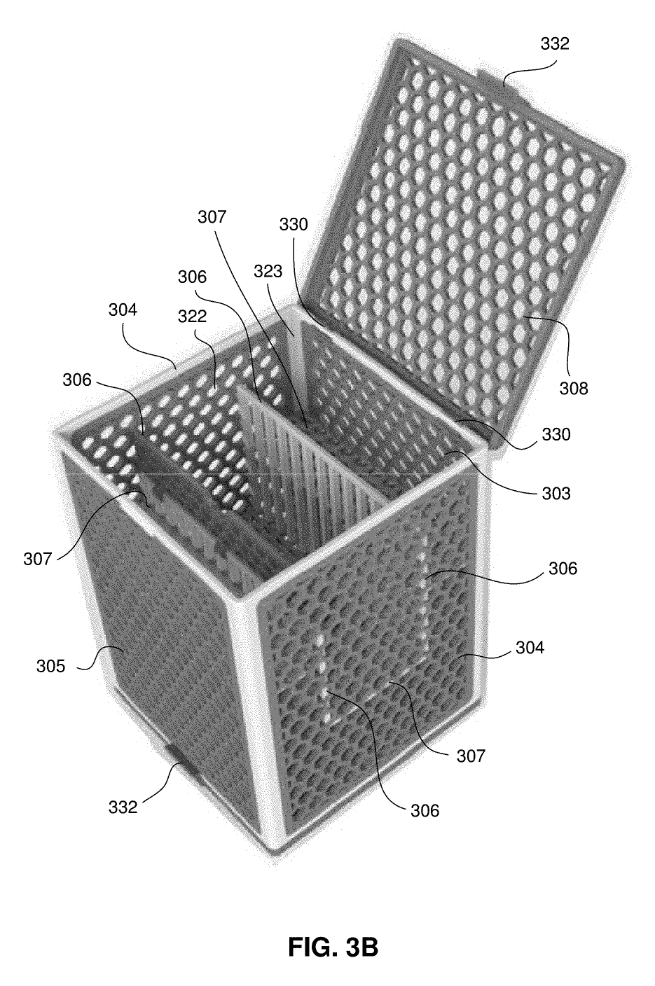

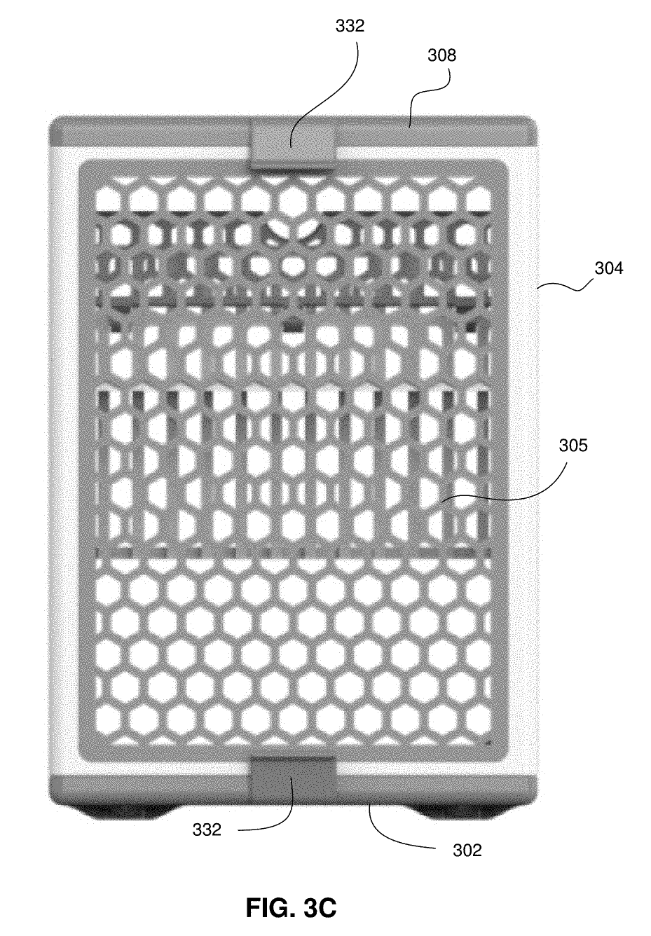

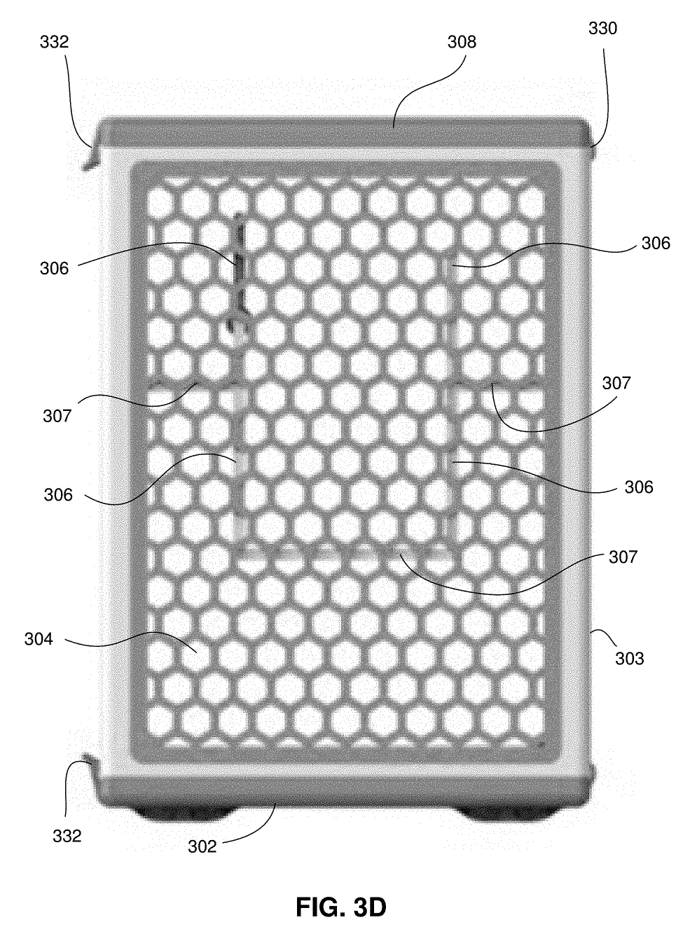

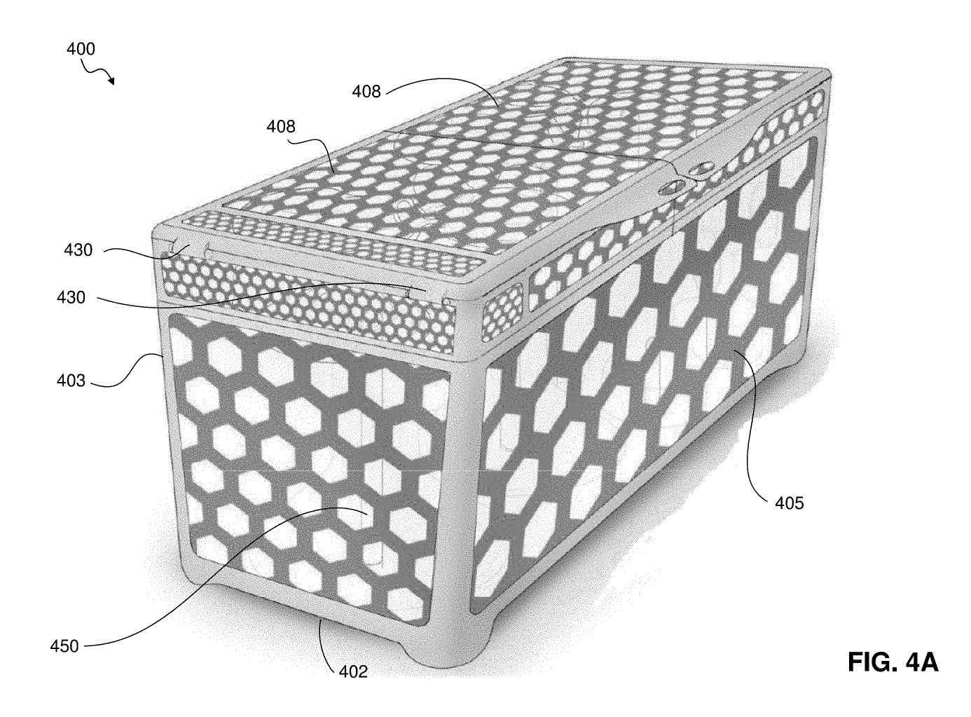

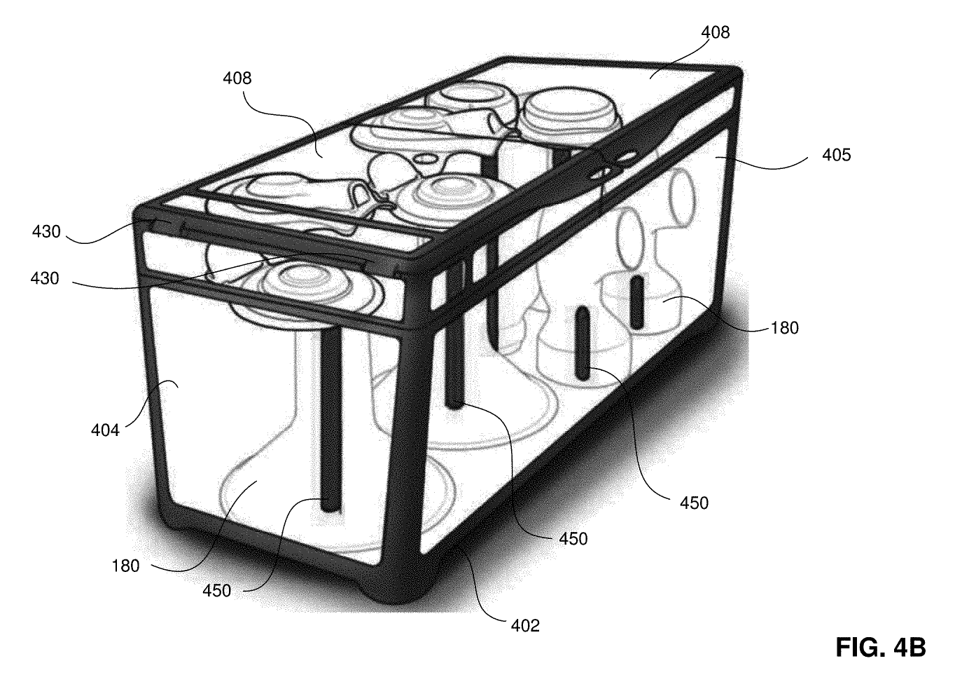

FIGS. 3A-3D and 4A-4B illustrate containers 300 and 400, respectively according to exemplary embodiments of the present disclosure. In one or more exemplary embodiments, the containers 300 and/or 400 can be rigid or semi-rigid containers. In an exemplary embodiment, one or more of the containers 300 and/or 400 are made of plastic, metal, a combination of plastic and metal, and/or one or more other materials or composites as would be understood by one of ordinary skill in the art. The containers 300 and/or 400 can be formed as baskets in one or more embodiments.

Turning to FIGS. 3A-3D, the container 300 includes a bottom panel 302, a back panel 303, multiple side panels 304, a front panel 305, and a top panel 308. The container 300 can include one or more divider panels 306, 307 that are configured to segment an interior space 320 formed by the panels 302, 303, 304, and 308 of the container 300.

In an exemplary embodiment, one or more of the exterior panels 302, 303, 304, 306, and 308 are formed to allow a washing fluid and air to enter and exit the interior space 320. For example, the one or more of the exterior panels 302, 303, 304, 306, and 308 can be formed as mesh walls having interconnected cell walls 310 and spaces 312 therebetween, but are not limited thereto.

In an exemplary embodiment, the interior divider panels 306, 307 can be any type of grating wall that divides the interior space 320 into a two or more interior sections 322, 323 and allows the water jets (e.g., washing fluid) to flow through the container 300 to clean parts in both interior sections. Examples of grating walls could include a mesh wall similar to the exterior panels or a grid wall with bars running between end slats, or the divider panel 306 could be a combination of these different types of grating walls.

The side panels 304, front panel 305, and the back panel 303 are connected to the bottom mesh panel 302 around a periphery of the bottom mesh panel 302 and extend away from the bottom mesh panel 302 to a top end 308. Accordingly, the side panels side panels 304, front panel 305, back panel 303 and the bottom panel 302 define an interior space 320. The top panel 308 serves as a lid to the container 300, being hingedly connected to one of the side panels (e.g., back panel 303) by one or more hinges 330 at the top end of the panel (e.g., top end of the back panel 303). The top panel 308 can be configured to rotate about the hinged connection between a closed (FIG. 3A) and an open (FIG. 3B) position. In an exemplary embodiment, the bottom panel 302 can also be configured to be hingedly connected to one of the side panels (e.g., back panel 303) by one or more hinges 330 similar to the connection of the top panel 308, and also rotate between a closed and an opened position. In an exemplary embodiment, the top panel 308 and/or the bottom panel 302 can include one or more fasteners 332 that are configured to releasably lock the top panel 308 and/or bottom panel 302 in a closed position. The fasteners 332 can be, for example, flexible tab that engages a protrusion on the panel, a snap clasp, a magnetic clasp, or another fastener as would be understood by one of ordinary skill in the art.

The divider panel(s) 306 fit within the interior space 320, extending from a location in the bottom half of the container 300 to a location close to the top panel 308. The divider panel 306 can be connected to a pair of the side panels 304 (and/or front 305 and/or back 303 panels) oppositely facing each other.

In an exemplary embodiment, the container 300 includes multiple vertical divider panels 306 and horizontal divider panels 307. As particularly illustrated in FIG. 3D, one of the horizontal divider panels 307 connects a pair of vertical divider panels 306, and another of the horizontal divider panels 307 connects one of the vertical divider panels 306 to the exterior panel (e.g., front panel 305). In this embodiment, the bottom panel 302 could also have a closed position and an open position as discussed above to permit access to opposite sides of the horizontal divider panels 307.

In an exemplary embodiment, the container 300 can be formed as a mesh panel construction with one or more side seams providing additional structural support similar to containers 100 and/or 200 described above. For example, one or more of the panels of the container 300 can be fabric panels. The fabric panels can be formed from a mildew-resistant, quick-drying textile, such as, for example, polyester, nylon or other synthetic fibers, but are not limited thereto. In an exemplary embodiment, the container 300 can be formed with a combination of rigid/semi-rigid panels and fabric panels.

FIGS. 4A and 4B illustrate a container 400 according to an exemplary embodiment of the present disclosure. In an exemplary embodiment, the container 400 includes a bottom panel 402, a back panel 403, multiple side panels 404, a front panel 405, and one or more (e.g., 2) top panels 408. In an exemplary embodiment, the container 400 can include one or more divider panels 406 (not shown) that are configured to segment an interior space 420 formed by the panels 402, 403, 404, and 308 of the container 300. In an exemplary embodiment, one or more of the panels can be formed as mesh walls having interconnected cells similar to container 300.

In an exemplary embodiment, the container 400 includes one or more elongated extensions or pegs 450 (more clearly shown in FIG. 4B) extending upwardly from the bottom panel 402. Similar to container 300, the top panels 408 are hingedly connected to the one or more of the panels (e.g., 402, 403, 404, 405) by one or more hinges 430. As shown in FIGS. 4A and 4B, the top panels 408 are hingedly connected to the left and right panels 404 and are configured to rotate between an open and closed position.

In an exemplary embodiment, the container 400 can include one or more horizontal divider walls that are hingedly connected to one or more of the panels (e.g., 402, 403, 404, 405) by one or more hinges within the interior 420 of the container 400. The horizontal divider walls can similar rotate between an open and closed position. When in a closed position, the horizontal divider walls can rest above the pegs 450 (and one or more parts 180 stored below and secured on the one or more pegs 450 and/or secured between two or more adjacent pegs 450. The horizontal divider wall can segment the interior space 420 of the container 400 into two or more interior sections. In an exemplary embodiment, as shown in FIG. 4B, the one more or of the panels 402, 403, 404, 405, 408 can be configured as an open panel without mesh walls having interconnected cells.

In an exemplary embodiment, the container 400 can be formed as a mesh panel construction with one or more side seams providing additional structural support similar to containers 100 and/or 200 described above. For example, one or more of the panels of the container 400 can be fabric panels. The fabric panels can be formed from a mildew-resistant, quick-drying textile, such as, for example, polyester, nylon or other synthetic fibers, but are not limited thereto. In an exemplary embodiment, the container 400 can be formed with a combination of rigid/semi-rigid panels and fabric panels.

In one or more exemplary embodiments, the containers are configured to be used to wash, dry, store, and/or carry breast pump parts 180, baby bottle parts, infant feeding parts or other kitchen and/or household items that are washed, dried, stored and/or carried. The exemplary embodiments describe containers that are configured to hold breast pump, bottle parts, and/or other articles separate from each other during cleaning so that the contained parts are sufficiently cleaned, while reducing the complexity, expense to manufacture, the spread of disease that can be harmful to the infant and/or mother.

The containers can have a defined bottom that allows it to stand upright with holes small enough to keep breast pump membranes (e.g., 0.75″ diameter) within the interior and large enough to allow for thorough washing and drying. The containers can be, for example, a basket or mesh bag, but are not limited thereto. The size of the container can be configured to fit one set of parts for a breast pump, but is not limited thereto. The container can keep the parts 180 properly oriented during the washing cycle and minimizes the space used during the wash, dry, store, and carry process.

### CONCLUSION

The aforementioned description of the specific embodiments will so fully reveal the general nature of the disclosure that others can, by applying knowledge within the skill of the art, readily modify and/or adapt for various applications such specific embodiments, without undue experimentation, and without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

What is claimed is:

1. A dishwasher container for breast pump parts, comprising:
a bottom fabric panel comprised of a first piece of mesh fabric, wherein the mesh fabric has a plurality of flexible fabric strands and a plurality of spaces between the flexible fabric strands;
a pair of side fabric panels connected to the bottom fabric panel at a bottom seam around a circumference of the bottom fabric panel and extending away from the bottom fabric panel to an opening at a top end, wherein the side fabric panels are comprised of a second piece of mesh fabric, wherein the side fabric panels are connected to each other through a pair of side seams, wherein the side seams extend from the bottom seam to the top end between the pair of side fabric panels, and wherein the pair of side fabric panels and the bottom fabric panel define an interior space;
a divider fabric panel within the interior space, wherein the divider fabric panel is comprised of a third piece of mesh fabric having a pair of sides, a bottom, and a top, wherein the divider fabric panel extends from the bottom at a first location in the interior space proximate to the bottom fabric panel to the top at a second location in the interior space proximate to the opening at the top end, and wherein the divider fabric panel divides the interior space into a pair of interior sections between the pair of sides; and
a closure secured at the top end around the opening, wherein the closure has a fastened configuration enclosing the interior space, and wherein the closure has an unfastened configuration allowing access to the interior space though the opening.

2. The container of claim 1, wherein the bottom fabric panel has a length greater than a width.

3. The container of claim 2, further comprising:
a second pair of side seams;
a pair of end fabric panels;
a third pair of side seams; a top fabric panel; and
a top seam, wherein the end fabric panels are connected between the side fabric panels through the pair of side seams and the second pair of side seams,
wherein the divider fabric panel is connected to the pair of side fabric panels through the third pair of side seams at an intermediate location between the end fabric panels,
wherein the top fabric panel is connected to the side fabric panels at the top end through the top seam,
wherein the divider fabric panel extends laterally across the bottom panel and the interior space, and
wherein the closure has a longitudinal axis generally perpendicular to a plane of the divider fabric panel.

4. The container of claim 3, wherein the top seam is further comprised of a pair of top lateral seams and a pair of top longitudinal seams.

5. The container of claim 2, wherein the divider fabric panel is connected to the pair of side fabric panels at the pair of sides through the pair of side seams, wherein the side seams are located at opposite ends of the length of the bottom fabric panel, and wherein the closure has a longitudinal axis generally aligned with a plane of the divider fabric panel.

6. The container of claim 1, wherein the side seams are further comprised of a bias tape, wherein the side seams have a greater rigidity than the side fabric panels and provide structural support to the side fabric panels.

7. The container of claim 6, wherein the side seams are further comprised of a bunching of mesh fabric.

8. The container of claim 4, wherein the pair of top lateral seams and/or the pair of top longitudinal seams are located on an exterior of the container.

9. A dishwasher container for breast pump parts, comprising:
a bottom panel, wherein the bottom panel is formed from a first mesh wall having a plurality of interconnected cell walls with a plurality of spaces between the cell walls;
a plurality of side panels connected to the bottom mesh panel around a periphery of the bottom mesh panel and extending away from the bottom mesh panel to a top end, wherein the each of the side panels is comprised of a second mesh wall, and wherein the side panels and the bottom panel define an interior space;
a top panel connected to the side panels at the top end, wherein the top panel is comprised of a third mesh wall, and wherein the top panel has a closed position and an open position; and
a divider panel within the interior space, wherein the divider panel is comprised of a grating wall having a pair of sides, a bottom, and a top, wherein the divider panel extends from the bottom at a first location in the interior space closer to the bottom panel than to the top panel to the top at a second location in the interior space proximate to the opening at the top end, wherein the divider panel is connected to a pair of the side panels oppositely facing each other, and wherein the divider panel divides the interior space into a pair of interior sections between the pair of sides.

10. The container of claim 9, wherein the bottom panel has a closed position and an open position.

11. The container of claim 9, wherein the grating wall is selected from a mesh wall, a grid wall, and a combination thereof.

12. The container of claim 9, further comprising a hinge, a plurality of vertical divider panels, and a plurality of horizontal divider panels, wherein the hinge connects the top panel to one of the side panels, wherein at least one of the horizontal divider panels connects a pair of vertical divider panels, and wherein at least another one of the horizontal divider panels connects one of the vertical divider panels to a side panel side extending between the panels oppositely facing each other.

13. The container of claim 9, further comprising a plurality of elongated pegs extending upwardly from the bottom panel.

14. The container of claim 13, wherein each of the plurality of elongated pegs is configured to securely hold one or more of the breast pump parts.

15. A method for cleaning breast pump parts, comprising:
loading one of more of the breast pump parts into a dishwasher container configured to securely contain the one more breast pump parts within an interior space of the dishwasher container;
loading the dishwasher container into a dishwasher that is operable to perform one or more dishwashing operations to automatically wash contents loaded into the dishwasher; and
controlling the dishwasher to perform one or more of the dishwashing operations.

16. The method of claim 15, wherein the dishwasher container comprises:
a bottom fabric panel comprised of a first piece of mesh fabric, wherein the mesh fabric has a plurality of flexible fabric strands and a plurality of spaces between the flexible fabric strands;
a pair of side fabric panels connected to the bottom fabric panel at a bottom seam around a circumference of the bottom fabric panel and extending away from the bottom fabric panel to an opening at a top end, wherein the side fabric panels are comprised of a second piece of mesh fabric, wherein the side fabric panels are connected to each other through a pair of side seams, wherein the side seams extend from the bottom seam to the top end between the pair of side fabric panels, and wherein the pair of side fabric panels and the bottom fabric panel define the interior space;
a divider fabric panel within the interior space, wherein the divider fabric panel is comprised of a third piece of mesh fabric having a pair of sides, a bottom, and a top, wherein the divider fabric panel extends from the bottom at a first location in the interior space proximate to the bottom fabric panel to the top at a second location in the interior space proximate to the opening at the top end, and wherein the divider fabric panel divides the interior space into a pair of interior sections between the pair of sides; and
a closure secured at the top end around the opening, wherein the closure has a fastened configuration enclosing the interior space, and wherein the closure has an unfastened configuration allowing access to the interior space though the opening.

17. The method of claim 15, wherein the dishwasher container comprises:
a bottom panel, wherein the bottom panel is formed from a first mesh wall having a plurality of interconnected cell walls with a plurality of spaces between the cell walls;
a plurality of side panels connected to the bottom mesh panel around a periphery of the bottom mesh panel and extending away from the bottom mesh panel to a top end, wherein the each of the side panels is comprised of a second mesh wall, and wherein the side panels and the bottom panel define the interior space;
a top panel connected to the side panels at the top end, wherein the top panel is comprised of a third mesh wall, and wherein the top panel has a closed position and an open position; and
a divider panel within the interior space, wherein the divider panel is comprised of a grating wall having a pair of sides, a bottom, and a top, wherein the divider panel extends from the bottom at a first location in the interior space closer to the bottom panel than to the top panel to the top at a second location in the interior space proximate to the opening at the top end, wherein the divider panel is connected to a pair of the side panels oppositely facing each other, and wherein the divider panel divides the interior space into a pair of interior sections between the pair of sides.
